(19) United States
(12) Patent Application Publication
(10) Pub. No.: US 2026/0130637 A1
(43) Pub. Date: May 14, 2026

(54) SYSTEMS AND METHODS FOR RAPID DIAGNOSTIC IMAGING OF PATIENTS SUSPECTED OF SUFFERING ACUTE ISCHEMIC STROKES

(71) Applicant: Circle Cardiovascular Imaging Inc., Calgary (CA)

(72) Inventors: Mayank Goyal, Calgary (CA); Johanna Ospel, Calgary (CA); Alireza Sojoudi, Calgary (CA)

(21) Appl. No.: 18/704,610

(22) PCT Filed: Oct. 26, 2022
(86) PCT No.: PCT/CA2022/051585

§ 371 Date: Apr. 25, 2024

Related U.S. Application Data
(60) Provisional application No. 63/272,409, filed on Oct. 27, 2021.

(51) Int. Cl. A61B 6/50; A61B 6/00; A61B 6/02; G06T 5/50; G06T 5/60; G06T 7/00; G06T 7/593; G06T 12/30; G06V 10/56; G06V 10/764

(52) U.S. Cl. CPC A61B 6/501; A61B 6/022; A61B 6/504; A61B 6/5217; G06T 5/50; G06T 5/60; G06T 7/0016; G06T 7/593; G06T 12/30; G06V 10/764; G06T 2207/10016; G06T 2207/10021; G06T 2207/10081; G06T 2207/10116; G06T 2207/20081; G06T 2207/30016; G06T 2207/30104; G06T 2207/30196; G06T 2211/404; G06T 2211/436; G06V 10/56

(57) ABSTRACT

Rapid diagnosis of acute ischemic stroke (AIS), and endovascular treatment—that is mechanical removal of the clot with endovascular devices—together leads to better outcomes. Systems and methods for decreasing the time to diagnosis are described that include imaging techniques conducted within an angiography suite using two rotating x-ray tubes that acquire x-ray images from a limited number of projection angles and that are processed to provide meaningful diagnostic data.

### FIELD

Rapid diagnosis of acute ischemic stroke (AIS), and endovascular treatment—that is mechanical removal of a blood clot with endovascular devices—together leads to better patient outcomes. Systems and methods for decreasing the time to diagnosis are described that include imaging techniques conducted within an angiography suite using two rotating x-ray tubes that acquire x-ray images from a limited number of projection angles and that are processed to provide meaningful diagnostic data.

### BACKGROUND

An angiography suite is a specialized imaging and treatment facility for conducting a wide range of minimally invasive procedures. The centerpiece of an angiography suite is an angiography machine on which specialized imaging and endovascular treatment is performed.

In the diagnosis and treatment of stroke, protocols referred to as direct-to-angio (DTA) are increasingly being implemented as an effective means to reduce the time normally associated with diagnosis and treatment of ischemic stroke. Specifically, DTA provides various advantages over traditional stroke protocols being practiced by bypassing traditional diagnostic steps that utilize imaging machines that can only be used for diagnosis (e.g. computed tomography (CT) or magnetic resonance imaging (MRI) scanners). By bypassing these initial imaging steps, the subsequent and additional step of patient transfer to the angiography suite is obviated.

In a traditional treatment protocol, when a patient arrives at a care facility and is suspected of having suffered a stroke, most care facilities would initiate protocols requiring the patient initially undergo a computed tomography (CT) scan in a standard or multi-use CT imaging suite, or an MRI scan. Typical scenarios using CT imaging are described as a CT scan. Generally, a CT scan is more common than MRI imaging; however, the same principles apply to MRI.

The typical multi-use CT scanner is a CT imaging device that is used to image a full range of body areas and is thus used to diagnose numerous conditions across all regions of the body.

The standard diagnostic CT machine directs a narrow beam of x-rays around a patient's body in a circle/helix via a rapidly rotating gantry. X-rays are emitted from an x-ray tube, and the x-rays pass through a region of interest of the body. The x-rays are received on the opposite side of the patient's body with a digital x-ray detector. Depending on the tissue, the x-ray beam will be attenuated based on the actual tissue the x-ray beam is passing through at a given time and position of the gantry arm. As the gantry is moving 360 degrees around the body, each x-ray beam is correlated to the time of the image and the angular position of the gantry, and through mathematical manipulation (i.e. post-processing), the multitude of data points are assembled into an image showing a slice of the body region (typically a “transverse” slice) at a particular level of the body.

Multiple rotations of the gantry at different positions/levels enable the construction of a series of spatially separated images slices of the underlying body tissues that can be displayed and/or assembled in a variety of ways to enable a radiologist to conduct diagnostic analysis of a particular region of the patient's body.

In addition to the basic operation of the CT machine, there are a wide range of techniques that a radiologist can employ which, depending on the suspected abnormality and the body regions being imaged, can be utilized to obtain greater precision and/or more relevant information from the images.

One specific technique utilized in a wide range of diagnostic procedures is a contrast CT scan. By injecting an iodinated contrast agent into the body shortly before the CT images are acquired, a contrast CT scan can specifically provide greater visibility to blood vessels when the images are acquired. Generally, as the contrast agent flow through the blood vessels, it attenuates x-rays more compared to the surrounding parenchyma, thus the blood vessels filled by contrast appear more dense, which provides a more effective differentiation between blood vessels and surrounding tissues through which the x-rays pass through more readily.

CT angiography (CTA) is particularly useful in the diagnosis of circulatory anomalies associated with ischemic stroke as the technique provides highly useful information about how contrast agent flows through affected and unaffected blood vessels/tissue which can be interpreted and used for effective diagnosis of ischemic stroke and enable planning of treatment procedures. Blockage of a brain vessel, which is the underlying cause of acute ischemic stroke (AIS), will appear as an abrupt filling defect of an otherwise contrast-filled vessel and lack of contrast in the brain tissue that is supplied by this vessel.

Each step of the acute ischemic stroke diagnosis and treatment workflow requires time including, for example, time for preliminary clinical examination, time to move the patient to and from different treatment areas (e.g. from the CT scanner where imaging is performed to a neuroangiography suite where treatment is performed), time for engagement with additional medical personnel including specialists, time to plan and execute additional diagnostic tests such as blood tests, time for image data processing and analysis, time to plan for treatment procedures (e.g. by choosing the appropriate endovascular treatment tools) and time for the actual execution of the treatment.

For patients suspected of suffering from AIS, “time is brain”. An average AIS patient with blockage of a large vessel loses on average 1.9 million neurons (brain cells) per minute. All steps that increase the time from stroke onset to treatment can substantially worsen patient outcome. By way of example, various combinations of the following steps may be followed for an ischemic stroke patient upon arrival at a treatment facility which as introduced above, each have a time component:
preliminary diagnosis based of patient symptoms (e.g. weakness, numbness etc.) by a first responder/physician to suspect potential stroke.
confirmation of preliminary examination by a stroke specialist and decision to conduct a CT scan to determine if AIS is truly present or whether there is an alternative diagnosis (e.g. hemorrhagic stroke).
movement of patient to a general CT suite, planning and execution of an initial CT scan.
post-process acquired CT images.
review of the images by the radiologist and complete initial diagnosis.
if AIS is suspected, plan and execute a CT angiography (CTA) scan (i.e. a contrast scan of the vessels that supply the brain), followed by additional processing time, review and diagnosis.
plan, execute and conduct additional CT studies such as a CT perfusion (CTP) scan or multi-phase (mCTA) scan (i.e. advanced imaging techniques that show brain tissue viability in AIS), followed by additional processing time, review and diagnosis.
decision to treat and move patient to a treatment location (e.g. the neuroangiography suite or “angio suite”).
Movement of the patient to the angiography suite and prepare and set-up for an endovascular procedure including choosing the appropriate devices, establishing endovascular access through puncture of a vessel, etc.).

Perfusion imaging is a popular imaging method for AIS since it can be displayed as color-coded maps of the brain. Occluded blood vessels cause stark abnormalities of perfusion that are displayed as wedge-shaped areas of different colors in perfusion maps, making vessel occlusions readily visible even for inexperienced operators. Further, perfusion maps allow operators to estimate how much of the diseased brain tissue is already irreversibly damaged and cannot be salvaged with treatment (“infarct core”), and how much of it is still salvageable with treatment (“tissue at risk”).

One specific issue during imaging is the amount of radiation received by a patient. CT imaging wherever performed relies on ionizing radiation and is proportional to the number of images acquired. In addition, within the angio suite, all endovascular tools are navigated under fluoroscopy control which also uses ionizing radiation. Hence, techniques that reduce the amount of radiation received by a patient are desirable while still providing effecting imaging.

Accordingly, there is a need for improved diagnostic and treatment protocols that reduce the time associated with the various tasks described above, including fully eliminating some steps, whilst reducing a patient's exposure to radiation.

As noted, protocols in some care facilities bypass the CT scanner and transfer the patient directly to the angio suite (DTA). At these facilities, the angio suite x-ray machine, which is normally only used to navigate endovascular catheters and other tools under fluoroscopy (x-ray guidance) during endovascular treatment is also used to acquire imaging.

In these DTA scenarios, the angio machine x-ray tube and detector rotate around the patient >360° (similar to a CT scanner) to produce images similar to CT which are referred to as “flat panel imaging”. This acquisition takes longer than a regular CT scan (several seconds) and also has a relatively high radiation dose. In addition, the image quality is poorer compared to a normal CT scan acquired with a regular CT scanner.

### SUMMARY

In accordance with the invention, there is provided a method of obtaining a series of perfusion images of a brain of a patient suspected of suffering from acute ischemic stroke (AIS) and having been injected with contrast dye, the method comprising the steps of: a) utilizing a neuroangiography machine having a first x-ray imaging system (FXIS) having a first x-ray emitter and a first x-ray receiver, the FXIS operatively positioned in a generally anterior/posterior position (AP) about the patient's head and a second x-ray imaging system (SXIS) having a second x-ray emitter and second x-ray receiver, the SXIS operatively positioned in a generally lateral position about the patient's head; b) obtaining a series of images from the FXIS and SXIS wherein the FXIS is activated to alternately move between first and second imaging positions and the SXIS is activated to alternately move between third and fourth imaging positions; c) repeating step b) over a plurality of time points between time=0 (t₀) to time=n (tₙ) and where t₀ generally corresponds to contrast arrival in upper cervical vessels and tₙ generally corresponds to contrast washout; and d) processing the images from steps b) and c) to create post-processed images showing a visualization of perfusion dynamics of the patient's brain.

In various embodiments:
step d) includes processing combinations of images from the FXIS and SXIS as a pair of images obtained within a time threshold and where if an area of affected tissue is detected, a relative depth of the affected tissue is estimated based on a stereoscopic offset analysis from a pair of images.
step d) further includes introducing images from step c) into an interpolation model and activating the model to match the images to a past image database derived from patients having been diagnosed with acute ischemic stroke by past computed tomography images and utilizing best-fit images from the past image database to interpolate for a greater number of image angles to improve the resolution of the post-processed images.
a first pair of images is acquired by activation of the FXIS to move to the first imaging position and subsequent movement of the FXIS to the second imaging position.
a second pair of images is acquired by activation of the SXIS to move to the third imaging position and subsequent movement of the SXIS to the fourth imaging position.
a first pair of images is acquired by activation of the FXIS to move to the first imaging position and movement of the SXIS to the second imaging position and images of the FXIS and SXIS are acquired simultaneously.
a second pair of images is acquired by activation of the FXIS to move to the third imaging position and movement of the SXIS to the fourth imaging position and images of the FXIS and SXIS are acquired simultaneously.
each of the first and second imaging positions and third and fourth imaging positions are about 15-30° apart.
the post-processed images are analyzed to classify affected brain tissue based on relative perfusion of the affected brain tissue on a color scale showing a range of colors between fully perfused brain tissue and dead brain tissue.
tₙ is 40-60 seconds.

In another aspect, the invention provides a method of training and utilizing an image-interpolation model to improve resolution of multiple flat-panel x-ray images derived from limited projection angles in a current patient, the multiple flat-panel x-ray images undergoing post-processing to assemble diagnostic images for diagnosis of acute ischemic stroke (AIS) in the current patient, the method including the steps of: a) obtain first multiple computed tomography angiography (CT) images, the first CT images being defined as full data set from a plurality of past patients having been diagnosed with AIS and where the first CT images includes images from multiple projection angles (e.g. more than 90 projection angles); b) obtain second multiple CT images, the second CT images being defined as a partial data set from a plurality of past patients having been diagnosed with AIS and where the second multiple CT images includes images from fewer projection angles (e.g. less than 10 projection angles); c) build a sinogram model for the first and second multiple CT images; d) train an interpolation model using the first and second multiple CT images to interpolate between projection angles of the second CT images based on determining a best-fit of a set of second multiple CT images to the first multiple CT images; and, e) introduce multiple current patient flat-panel x-ray images into the model and analyze to determine a best-fit with the first multiple CT images to create interpolated images from the current patient.

In another aspect, the invention provides a method of operating a neuroangiography machine having a first x-ray imaging system (FXIS) having a first x-ray emitter and a first x-ray receiver and a second x-ray imaging system (SXIS) having a second x-ray emitter and second x-ray receiver, comprising the steps of: a) moving the FXIS to a first position in a generally anterior/posterior position relative to a patient's head; b) moving the SXIS to a second position in a generally lateral position relative to a patient's head; c) activating the FXIS to alternatively move between a first imaging position and a second imaging position through an arc about the anterior/posterior position; d) simultaneously activating the SXIS to alternatively move between a third imaging position and fourth imaging position through an arc about the lateral position and, e) obtaining x-ray images at each of the first, second, third and fourth imaging positions.

In various embodiments, movement of the FXIS and SXIS is coordinated to enable acquisition of images simultaneously from the FXIS and SXIS when the FXIS is at the first or second imaging position and the SXIS is at the third or fourth imaging position.

In another embodiment, steps c) to e) are repeated over a plurality of time points between time=0 (t₀) to time=n (tₙ) and where after injection of contrast agent into the patient, t₀ generally corresponds to contrast arrival in upper cervical vessels and tₙ generally corresponds to contrast washout.

In various embodiments each of the first and second imaging positions and third and fourth imaging positions are about 15-30° apart and/or tₙ is 40-60 seconds.

In various embodiments, the method includes the step of post-processing images to classify affected brain tissue based on relative perfusion of the affected brain tissue on a scale discerning between fully perfused brain tissue and dead brain tissue.

### BRIEF DESCRIPTION OF THE DRAWINGS

Various objects, features and advantages of the invention will be apparent from the following description of particular embodiments of the invention, as illustrated in the accompanying drawings. The drawings are not necessarily to scale, emphasis instead being placed upon illustrating the principles of various embodiments of the invention. Similar reference numerals indicate similar components.

FIG. 1 is an end-view sketch of typical angiography suite equipment in accordance with one embodiment of a method of the invention.

FIG. 2 is a side-view sketch of an angiography suite equipment in accordance with one embodiment of a method of the invention.

FIG. 3 is a sketch showing representations of typical images acquired through anterior/posterior (AP) and lateral (L) imaging in a patient having affected tissue.

FIG. 4 is a sketch showing principles of stereoscopic imaging to obtain a determination of the depth of an area of interest (e.g. affected tissue).

FIG. 5 is a sketch showing how AP and L images may be assembled to determine depth of an area of interest.

FIGS. 6 and 6A are sketches showing how the AP and L systems may be moved from a neutral position to imaging positions to take sequential images in accordance with two embodiments of the invention.

FIG. 7 is a sketch showing representative relative flow rates of contrast through unaffected and affected tissues.

FIGS. 8, 8A, 8B and 8C show A) the variation in projections based on projection angle; B) a full sinogram; C) a representative partial sinogram and D) representative projections in the space and Fourier domains.

FIG. 9 is a flowchart showing a generalized process for building, training and utilizing a model enabling interpolation of limited angle images from a current patient to output interpolated images for a current patient.

### DETAILED DESCRIPTION

### Rationale and Language

The inventors have recognized that direct-to-angio (DTA) and angio suite imaging, which utilizes angio suite x-ray equipment, can be improved by conducting unique movements of the imaging equipment in a manner that provides various advantages over currently used on-table DTA imaging, including the acquisition time and amount of radiation received by a patient during diagnostic imaging.

With reference to the figures, systems and methods for conducting diagnostic imaging in an angiography suite are described. Within this description, all terms have definitions that are reasonably inferable from the drawings and description, with the language used herein to be interpreted to give as broad a meaning as is reasonable. Within this application, reference is made to various numbers and number ranges. Numbers or number ranges are to be interpreted with the understanding that numbers are defining possible boundaries or variables related to particular features described herein. Boundaries are not necessarily fixed and may be affected by relationships with one or more other features. Thus, use of terms like “about” or other modifiers in this description are intended to provide allowance for the potential interplay of variables or features with respect to one another and should be interpreted in that light. Features described herein are understood to provide collective functionality. At a minimum, numbers are to be interpreted having regard to their significant digits.

### Introduction

For the purposes of illustration, components depicted in the figures are not necessarily drawn to scale. Instead, emphasis is placed on highlighting the various contributions of the components to the functionality of various aspects of the invention. A number of possible alternative features are introduced during the course of this description. It is to be understood that, according to the knowledge and judgment of persons skilled in the art, such alternative features may be substituted in various combinations to arrive at different embodiments of the present invention.

### Angiography Suite and Equipment

The imaging equipment in an angiography suite (“angio suite”) is primarily designed for the specific imaging requirements for the treatment of a range of circulatory disorders. For the purposes of this description, the angio suite is described in relation to the diagnosis and treatment of acute ischemic stroke (AIS).

As noted above, an angiography suite is a specialized imaging and treatment suite within a treatment facility designed to enable specialized imaging prior to and during endovascular procedures utilizing endovascular equipment (EE). EE generally refers to a wide range of catheters, wires, microcatheters, stents and other devices that can be moved from an entry point through the circulatory system during an endovascular procedure.

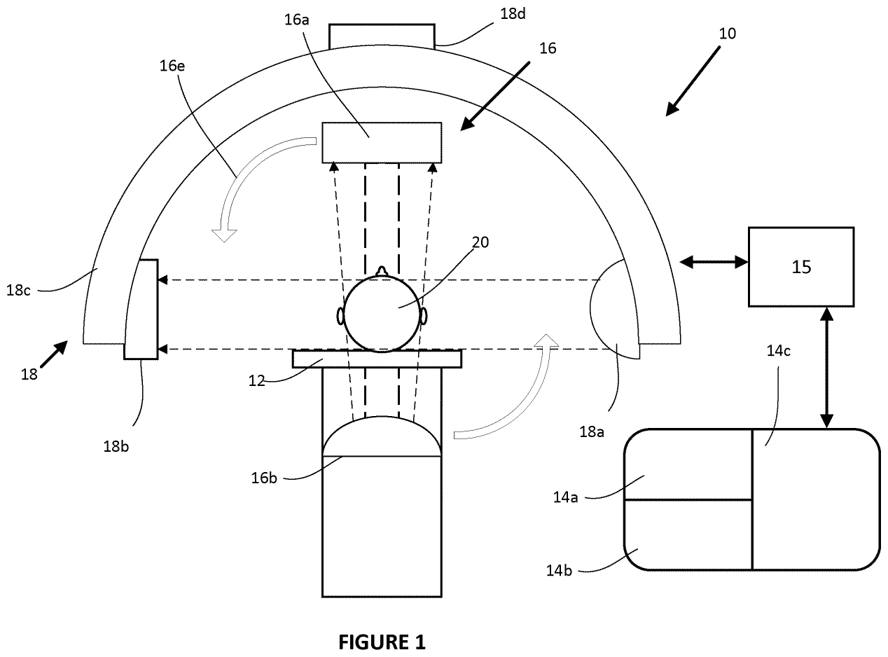

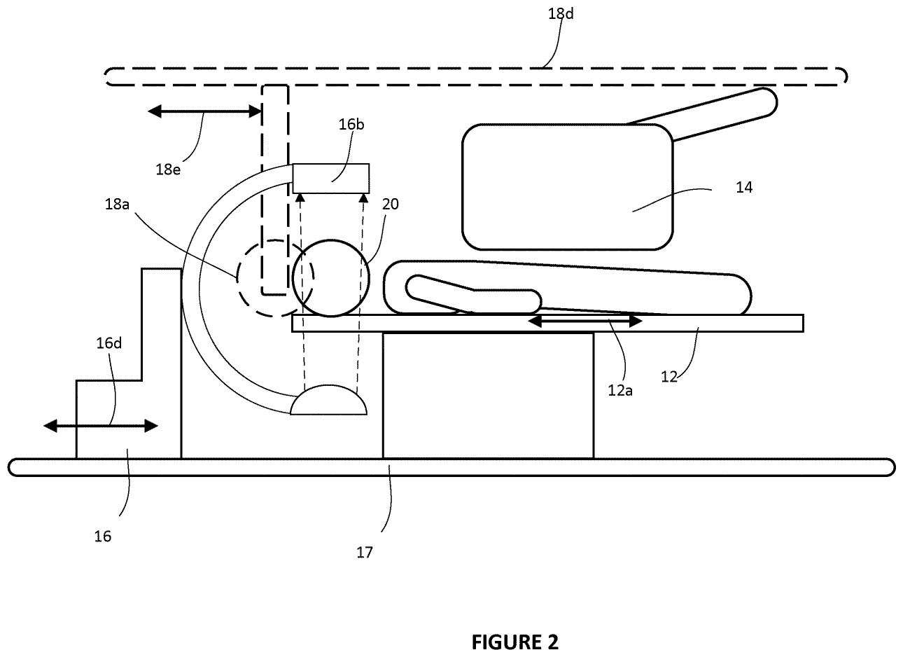

As shown in FIGS. 1 and 2, the typical angio suite includes a combination of imaging and treatment equipment 10 including a patient treatment table 12 together with computer 15 and display equipment 14 that allows a surgeon to perform both diagnostic imaging of a patient and real-time imaging during an endovascular procedure. The imaging equipment generally includes x-ray equipment allowing the operator to conduct two main types of imaging including cross-sectional flat panel imaging and real-time fluoroscopic (x-ray) imaging during a procedure to visualize EE.

The angio suite includes two x-ray systems/tubes that can be operated to conduct cross-sectional flat panel imaging and real-time fluoroscopy imaging. Herein, these x-ray systems are referred to as anterior/posterior (AP) x-ray tube/system 16 (AP system) and a lateral (L) x-ray tube/system 18 (L system).

The AP system can be operated to spin through approximately 200 degrees (either in a rotational fashion or in figure-8 shaped/butterfly-shaped movements) enabling acquisition of images for various diagnostic CT imaging techniques including non-contrast cross-sectional flat panel imaging, flat panel contrast cross-sectional angiography imaging following a contrast injection (either single-phase or multi-phase angiography), and flat panel perfusion imaging.

In flat panel perfusion imaging, a single rotational imaging is typically performed over 5-20 seconds. This can be done without contrast to generate images that are similar to a non-contrast CT scan. However, the resolution and quality of these images is typically worse compared to a non-contrast CT scan. Thus, differentiating the brain tissue types and detecting early signs of AIS is challenging.

A map similar to a perfusion imaging map can be made using a single rotational data acquisition acquired over approximately 8 seconds. However, this has many limitations. Most importantly, 8 seconds is not enough for blood to completely wash in and out of the brain. Therefore, it is not possible to truly generate cerebral blood flow (CBF), and cerebral blood volume (CBV) maps.

Some have tried to acquire repeated images over a time span of approximately 45 seconds to show contrast arrival, distribution and washout in brain vessels and brain tissue in real time. However, given that one acquisition is typically about 5-8 seconds, this severely limits temporal resolution. In addition, it substantially adds to the radiation exposure. It also increases vulnerability to patient motion; particularly since many AIS patients are very ill and unable to lie still.

In flat panel perfusion imaging, a total of 200-600 images are usually acquired, with a typical radiation dose of >200 mGy (Fiorella D, Turk A, Chaudry I, Turner R, Dunkin J, Roque C, et al. A prospective, multicenter pilot study investigating the utility of flat detector derived parenchymal blood volume maps to estimate cerebral blood volume in stroke patients. J NeuroInterventional Surg. 2014; 6(6):451-456. doi: 10.1136/neurintsurg-2013-010840). This radiation exposure is further increased if the acquisition is over 45 seconds.

The AP system may also be held statically for imaging in the AP directions for fluoroscopic visualization of EE during treatment. For this procedure, the AP system 16 is positioned statically above and below the patient as shown in FIGS. 1 and 2. The L system 18 is generally in a fixed position in a lateral position on both sides of the patient's head as shown in FIG. 1 and is typically used only for EE visualization. As catheters are advanced from the patients arm or groin towards the brain, the AP and L tubes can be moved along the patient's long axis and rotated slightly in between fluoroscopic image acquisitions to keep the EE in the field of view.

As shown, the AP tube 16 includes an x-ray emitter 16a and receiver 16b and the L tube includes an x-ray emitter 18a and receiver 18b. Both the AP and L systems can be positioned relative to a body region of interest such that the x-rays of each tube pass through the body region to a corresponding receiver. Each receiver is typically a 48 cm by 48 cm receiver enabling imaging of the entire brain in a single image. Both the emitters and receivers are connected to computer control systems 15 that control activation and movement of each emitter and receiver and that provides for the collection and processing of data from each. Video displays 14 enable the display of imaging data (both in real-time and as a repeat display of stored previously acquired image sequences).

Each of the AP 16 and L 18 systems are generally operable independent of one another. That is, each can be spatially moved relative to one another and relative to the patient and can be independently operated to obtain a variety of images. Generally, the L system must be fully moved out of the way during flat-panel imaging when only the AP system is used, which is then rotated >360° around the patient.

In one example, the L tube system 18 includes a c-arm 18c supporting the x-ray emitter 18a and detector 18b suspended from a ceiling gantry 18d that can be moved parallel to the longitudinal axis of the treatment table as shown by the double arrowed lines 18e in FIG. 2. Longitudinal movement of the gantry allows the surgeon to position the L tube system 18 where desired for imaging a particular body region (e.g. the head 20 of a patient). In addition, the table 12 may also be positioned along the longitudinal access and may also be moved parallel to the longitudinal axis as shown by line 12a.

The AP system 16 is typically mounted on a c-arm 16c and stand 16d on the floor 17. The c-arm 16c can be made to rotate in a circular/helical motion around the body as shown by arrows 16e. The AP system may also be moved parallel to the longitudinal axis as shown by line 16e.

As noted, each of the AP system 16 and L system 18 can be moved out of the way of each other in order that there is no overlap between each system as may be required at various times and specifically when the AP system is fully rotated for flat panel imaging.

### Representative Treatment Scenarios

In a typical treatment scenario with a patient suspected/diagnosed as suffering AIS, the patient is delivered to the treatment table 12, sedated and partially immobilized.

### Separate Imaging and Earlier Diagnosis

If previous diagnosis has been conducted via CT imaging (or MR imaging), the surgeon will start preparing for a planned procedure, for example an M1 large vessel occlusion (LVO) thrombectomy via a femoral artery access route.

Groin puncture is completed and the desired EE advanced to the aortic arch. Upon reaching the aortic arch, the EE is advanced into the appropriate cervical artery utilizing real-time x-ray fluoroscopy and contrast injected via the EE. That is, the surgeon will have positioned the AP and L systems orthogonal to one another as shown in FIG. 1. After injecting contrast, the AP and L systems are selectively turned on to display AP 14a and L 14b orientation images in real time on the display system 14. Generally, the AP images 14a show the position of the EE from a side (lateral) view and the L images 14b show the anterior/posterior position of the EE. Other images 14c, for example from diagnostic studies may also be displayed to help the surgeon orient him/herself. Selective use of the AP and L imaging systems enables the surgeon to complete the procedure with contrast being injected as required.

### Direct to Angio (DTA) Protocol

In a DTA protocol, diagnostic imaging studies such as CT, single or multiphase CT angiography, and CT perfusion are not performed in a separate imaging suite and the patient suspected of having suffered a stroke is transported directly to the angio suite upon arrival at the treatment facility (direct to angio workflow). The first imaging that is performed is flat panel imaging on the angio table, as described below.

As noted, the AP system 16 is rotatable and accordingly can be used to obtain >360° rotational flat panel studies similar to any of the above-mentioned CT studies through rotation of the AP system around the head. Generally, to conduct diagnostic flat panel imaging, the L system 18 is fully withdrawn to allow the AP system 16 to be rotated around the patient's head. After imaging and analysis, the AP and L systems are positioned as shown in FIG. 1 for the treatment to be conducted.

Diagnostic flat panel images are collected from the c-arm rotating >360 degrees around the patient. In most cases, the initial scan would be non-contrast flat panel imaging to rule out alternative diagnosis such as brain bleeds (so-called hemorrhagic stroke).

Modified perfusion imaging studies can be conducted in the angio suite where CT perfusion look-alike flat panel images are collected at a rate of typically 5-10 images/second over an imaging period of approximately 5-8 seconds. However, this is grossly insufficient compared to the approximate time it will take contrast to fully traverse the cranial arteries, brain tissue and venous drainage system with a large vessel occlusion and contrast being held up on the ipsilateral side.

In other protocols, multiphase angiography flat panel imaging is utilized which is a diagnostic imaging technique that obtains a discrete number of phases (typically 3-6) of images over the imaging period. Multiphase angiography flat panel images are generally acquired at specific times having regard to the flow of contrast into the brain and through the two hemispheres (typical phases include the arterial phase, peak-venous phase and late-venous phase).

### Non-Rotational Perfusion Imaging (NPI) in Angio Suite

In accordance with the invention, various embodiments of Non-Rotational Perfusion Imaging (NPI) are described utilizing AP and L imaging systems for diagnosis. As described herein, NPI imaging reduces radiation to the patient over the course of the imaging while obtaining sufficient data to construct perfusion maps that enable differentiation of the spatial location of contrast density within each image at each time point and specifically the identification of areas that are affected by the vessel occlusion.

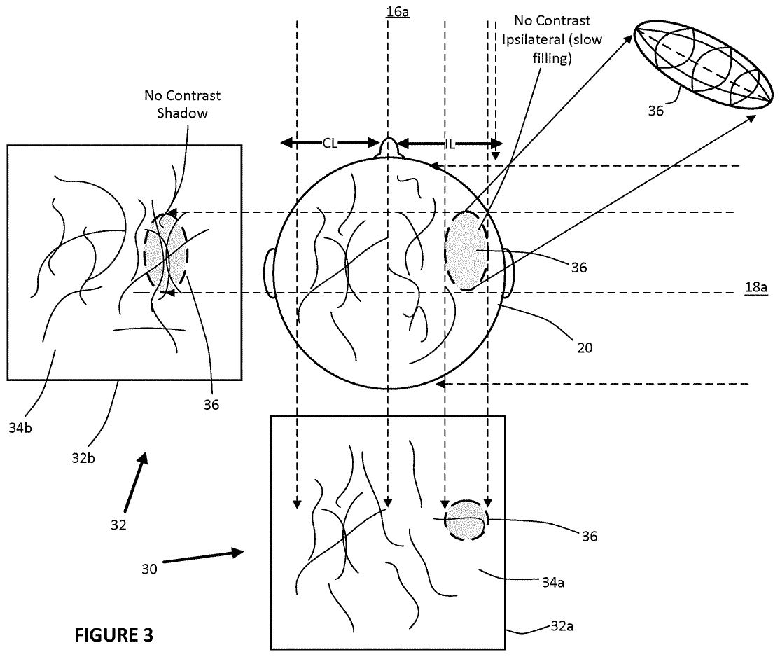

As shown in FIG. 3, AP images 30 and L images 32 obtained with the AP and L systems in their static positions are different given the angular position from which each image is acquired and the orientation of the brain during image acquisition. For the purposes of describing images 32a and 32b, it is to be noted that the AP and L images are shown in the plane of the page but it is to be understood that each image represents an image captured with the receiver 90 degrees to the plane of the page.

While images 32a, 32b are useful for treatment, they do not provide sufficient data for diagnosis as a result of the fixed image angles and the shadowing/masking effects of tissues having contrast vs. not having contrast being in front or behind each other.

For example, FIG. 3 shows AP and L images at a time shortly after contrast flooding the brain. Unaffected tissue 34a, 34b is opacified on both the ipsilateral (IL) and contralateral (CL) sides as shown by representative blood vessels. A region/volume of affected tissue 36 is shown on the right side. In the AP image 32a, the affected tissue 36 may be spatially discernable from the unaffected tissues, as only a single vessel projects onto the affected area. The vessel density of the affected area 36 in the AP image 32a is overall much reduced compared to healthy tissue 34a. However, given the number of opacified vessels behind the affected tissue on the L image 32b, spatial discernment of the affected tissue is difficult against the opacified vessel background on the lateral view. As such, and as described in greater detail below, static use of the AP and L systems cannot provide sufficient discernment to affected vs. unaffected tissues to be useful for diagnosis. Moreover, as noted above, the AP system 16 cannot rotate through its normal arc without collision with the L system 18.

In accordance with the invention, methods of simultaneously operating the AP and L systems in a coordinated fashion to collect diagnostic images to spatially separate affected from healthy tissue are described.

Movement of the AP and L systems is conducted in order that the two systems don't collide with one another while enabling acquisition of diagnostic images from more angles than when the AP and L systems are operated statically during fluoroscopy. As described, images collected enable discernment of regions of affected and unaffected tissues through stereo-imaging techniques.

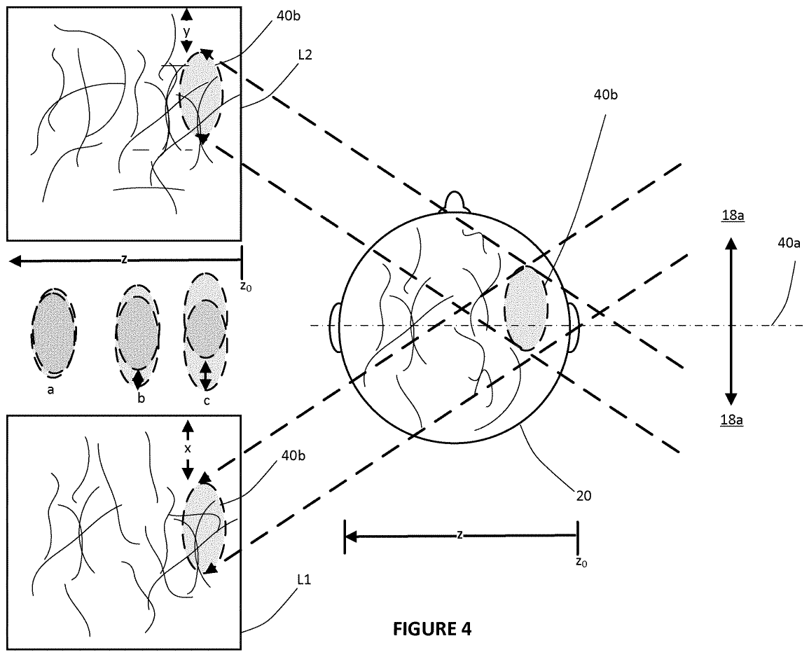

With reference to FIG. 4, the general principles of operation are described.

A patient's head 20 is being imaged. A lateral axis 40a showing a normal static position of an L system is shown for reference. An area of affected tissue 40b (showing no contrast; shown in the figures with shading) is on the right side and the left side (contralateral side) shows normal filling. The L system 18 is sequentially moved to image the head at two angles, approximately ±15-30° to create images L1 and L2. Each image on its own does not provide sufficient information to determine a depth z relative to z₀. However, as the two images are angularly displaced, the positions of L1 and L2 projections of the affected tissue 40b are laterally displaced as shown by distances x and y on the two images.

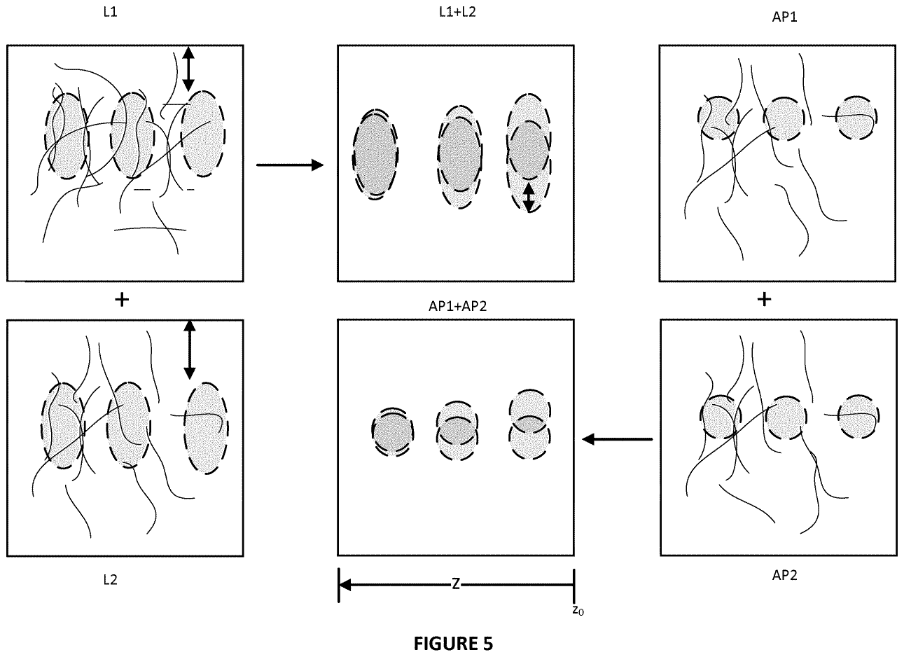

The AP system 16 can be similarly operated and moved to obtain AP1 and AP2 images at similar offsets to a vertical axis (e.g. about)+15-30°. The combination of the 4 images enables determination of the relative depth of the affected tissue in both directions and, hence, the specific location within the patient's brain as shown in FIG. 5.

By overlaying the images of the affected tissue as shown in FIGS. 4A and 5, the relative lateral depth z (relative to a defined z₀) of the affected tissue can be determined by the lateral offset a, b, c of overlaid L1 and L2 images. That is, the lateral offset a, b, c decreases (see FIG. 4) with greater lateral depth allowing a determination of depth (i.e. distance of the affected tissue from the x-ray emitters) from the two images.

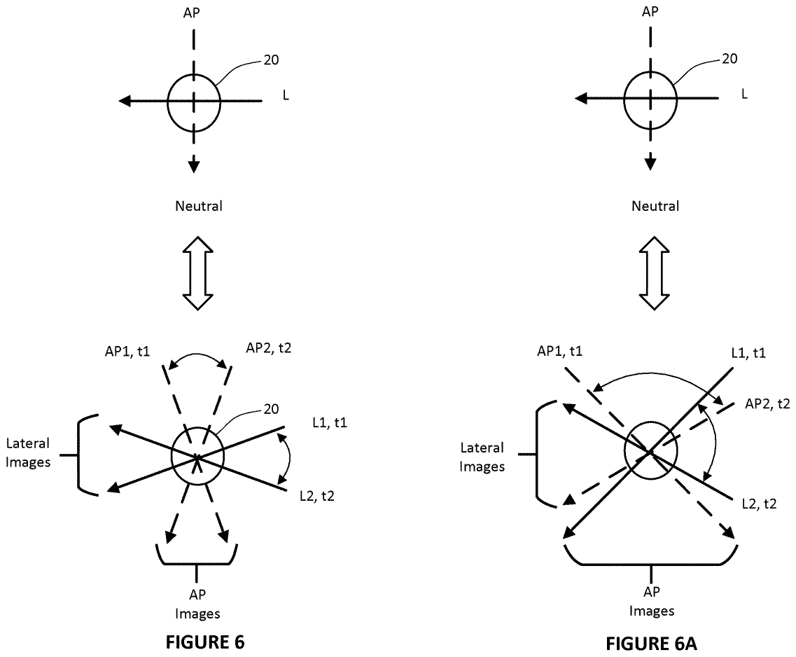

The AP and L systems can be sequentially operated as shown in FIGS. 6 and 6A where L1, L2, AP1 and AP2 images can be collected at representative positions relative to neutral under two operating scenarios. As shown in FIG. 6, the L1 and L2 images will be collected to create an image pair for a lateral image and AP1 and AP2 images will be collected to create an image pair for an AP image. As shown in FIG. 6A, both a lateral and AP set of images is acquired from one L and AP image.

Generally, as it takes time (about 0.5-1.0 sec) for the AP or L systems to move through a 30-60° arc, images taken sequentially from each system will be temporally separated. That is, L1, L2 and AP1, AP2 images will be time-separated by approximately 1 second.

Under various post-processing scenarios, it may be favorable to obtain each set of lateral and AP images at the same time; hence, if physical space and projection angles permit, the movement of the AP and L systems as per FIG. 6A may be preferred.

### Imaging Protocol

To obtain a series of diagnostic flat panel perfusion images, after the patient is immobilized, the operator/surgeon will initiate contrast injection. Images may be collected according to known triggering procedures, such as contrast entering the upper cervical vessels. Movement of the AP and L tubes is coordinated by system software to prevent collision with the other.

In a typical scenario where a large vessel occlusion is suspected, the expected time period for contrast to fully flush through the arteries, brain tissue, and draining veins of the brain on both the IL and CL sides will be about 45 seconds.

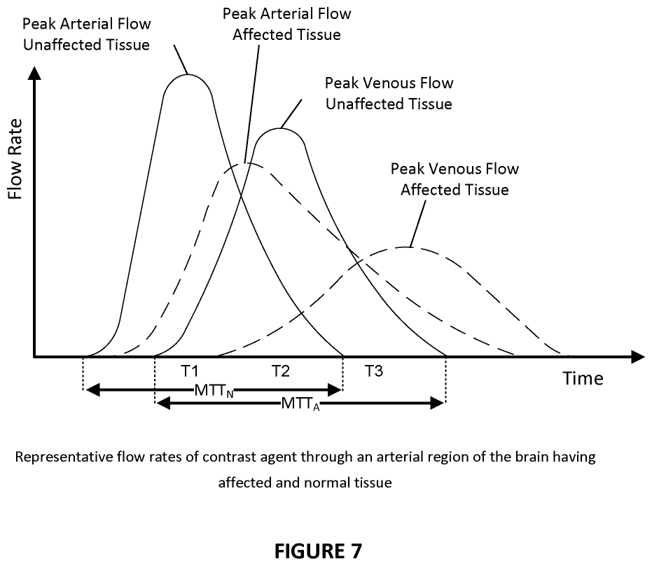

Initially, images will generally be taken as rapidly as possible from each of the AP1, AP2, L1 and L2 positions, and preferably at a rate of greater than 1 set of images/second. This first stage of images would be taken from t=0 to about t=5 seconds so as to capture images immediately before, during and after peak opacity of unaffected tissue as shown in FIG. 7. Over the next five seconds, which may correspond to contrast flushing out of the unaffected tissue through draining veins and a slow rise of contrast in affected tissues (in which contrast flooding is slower because of the blocked brain vessels which cause the AIS), the image rate may be slowed down as the rate of change of density in affected tissues is lower and thus spread out over a greater time period. After contrast has flowed out of the arterial system, the image acquisition rate may be slowed further (e.g. one set of images/2 seconds) for the remainder of the acquisition time.

As shown in Table 1, representative image acquisition rates are shown assuming both AP and L images are being acquired. 2 sets of images=4 images.

TABLE 1
Representative Image Acquisition

| Time | Image Rate | Total Time (secs) | Total Images |
|---|---|---|---|
| 0-5 | 1-2 sets of images | 5 | 10-20 |
| | per sec = 2-4 | | |
| | images per second | | |
| 6-15 | 1 set of images per | 10 | 20 |
| | sec = 2 images per | | |
| | second | | |
| 16-45 | 1 set of images per | 35 | 18 |
| | TWO seconds = 0.5 | | |
| | images per second | | |
| Total: | | | 48-58 images |

Accordingly, if a total of approximately 50-60 images in total are taken, this is substantially lower than a typical CTA or CTP study where 200-600 images may be acquired over the same time period, particularly, if due to patient motion, the scan must be repeated. Thus, the total radiation to the patient is substantially lower.

The imaging procedure may also be repeated during the procedure as may be required. That is, because imaging can be conducted without having to move the L system out of the way, new diagnostic imaging can be initiated at any time. This is particularly useful if for example, a large blood clot has been removed and the surgeon wants to confirm that no pieces of the blood clot broke off and have resulted in small peripheral occlusions.

Depicting the perfusion abnormalities in cases in which a large blood clot broke off into smaller clots can be helpful for detecting downstream occlusions and resulting perfusion deficits caused by those broken off clot fragments, and for deciding whether the small clot fragments can be and should be treated with endovascular tools or not. With flat panel perfusion imaging as it is used currently however, the L tube first has to moved out of the way, resulting in a time delay until new perfusion imaging acquisition can be started. With the NPI technique, the L tube can remain where it is and perfusion imaging to show perfusion abnormalities related to small broken off blood clots can be initiated right away, thereby minimizing treatment delays.

Another option at this stage is the option of injecting directly into the internal carotid artery (i.e. direct injection into the vessel that has is blocked further downstream). This has the advantage of achieving a much higher contrast density because the contrast is injected directly into the affected vessel, and not into the systemic vasculature, and it is therefore much less diluted. This results in better signal to noise ratio. However, this does not allow complete filling of the collateral circulation, because only the collateral vessels originating from the internal carotid artery are filled, but not collateral vessels supplying the affected brain tissue that originate from other arteries, e.g. from the internal carotid artery of the other, healthy side, or from the vertebral arteries, which are other major arteries supplying the brain tissue. This means that one is missing important information on collateral filling from other major brain vessels.

### Image Acquisition and Post Processing

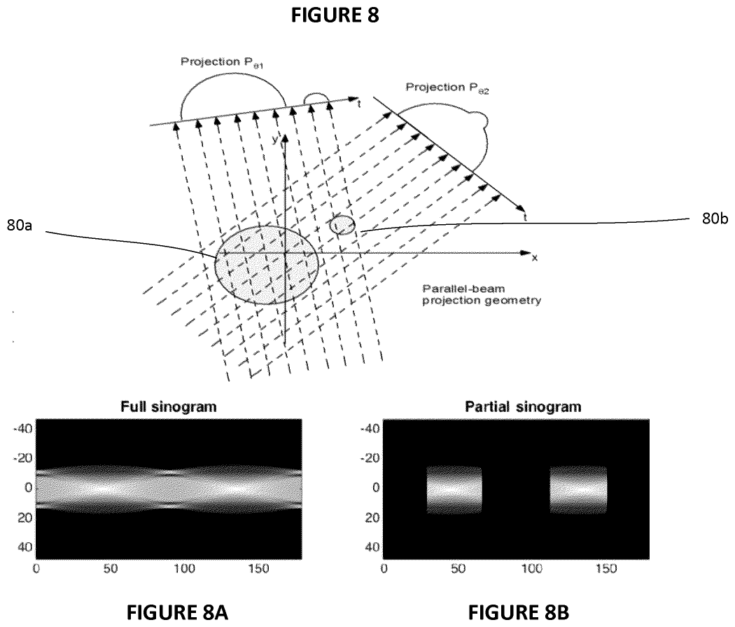

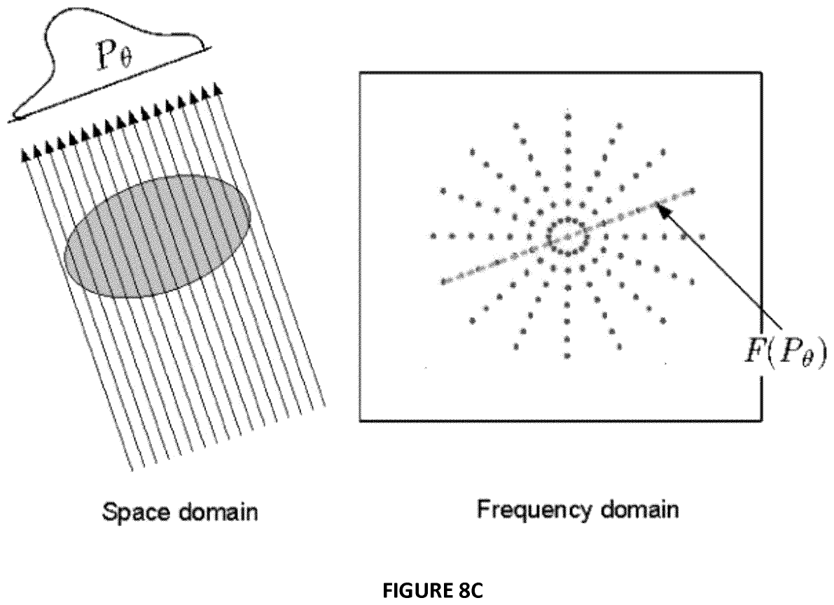

As noted above, acquisition of flat panel images is typically obtained by rotating an x-ray tube about a 200 degree (either in exact circles or butterfly-shaped/figure 8-shaped movements) to obtain a plurality of projections.

Post processing of raw image data enables transformation of the raw image data into useful displays including for example classifying affected brain tissue based on relative perfusion of brain tissue. Post processing of the images described above may include incorporating a scale such as a color scale showing a range of colors between fully perfused brain tissue and dead brain tissue.

Generally, projections over each 180 degrees of rotation are used for separate image processing. As shown in FIG. 8, an object being scanned will produce a different projection dependent on the angle that it is being scanned. Two circular objects 80a, 80b being scanned from different angles will result in 2 spatially separated semi-circles at θ1 and combined semi-circles at θ2. Projections across 180 degrees where images are taken without angular gaps enables assembly of a full sinogram (FIG. 8A) whereas projections with angular gaps enables assembly of partial sinograms (FIG. 8B).

FIG. 8C shows a Fourier slice projection. In summary a projection of a 2D object at an arbitrary angle produces a 1D signal. The Fourier transform of this 1D signal is a 1D frequency function that can be shown as a line in the 2D Fourier domain of the 2D image with the same angle. Thus, by collecting multiple projections at different angles, the 2D Fourier domain of the image is filled line by line. The Fourier space shows that the center is oversampled and the boundaries are under sampled where the center of the k space represents the low frequencies and the outside represents the high frequencies of the image.

Image assembly typically utilizes filtered backprojection methodologies including Fast Fourier Transform (FFT) of the image projection (at each angle), followed by filtering, and Inverse Fast Fourier Transform (IFFT) to create filtered backprojection functions that when summed from a plurality of angles generally improves the clarity of object edges proportional to the number of projection angles.

Backprojection is substantially the reverse operation of a forward projection. A backprojection from a detector function at a single angle results in a smeared representation of the image. As additional backprojection angles are added, the clarity of the object is improved but ultimately limited due to the low frequencies vs. high frequencies densities derived/mapped by transformation into the Fourier domain as noted above. That is, backprojections derived from a Fourier domain representation of low and high frequencies shows that low frequencies density is higher in the Fourier domain (i.e. generally smooth inner surfaces of an object) vs. high frequencies density which are lower (i.e. generally boundaries or edges of the object).

Filtering of the backprojection functions enables suppression of some the low frequencies and amplification of the high frequencies, wherein when subject to Inverse Fast Fourier Transform (IFFT) results in a new detector function that is subsequently backprojected over the domain to provide improved imaging of object edges particularly as the sampling angles are increased.

### Machine Learning

In one embodiment, to the extent that the limited projection angles described above do not enable adequate discernment of affected vs. unaffected tissue, together with the stereoscopic techniques described above, partial sinograms derived above from the limited projections can be interpolated using model development and training to interpolate current patient data.

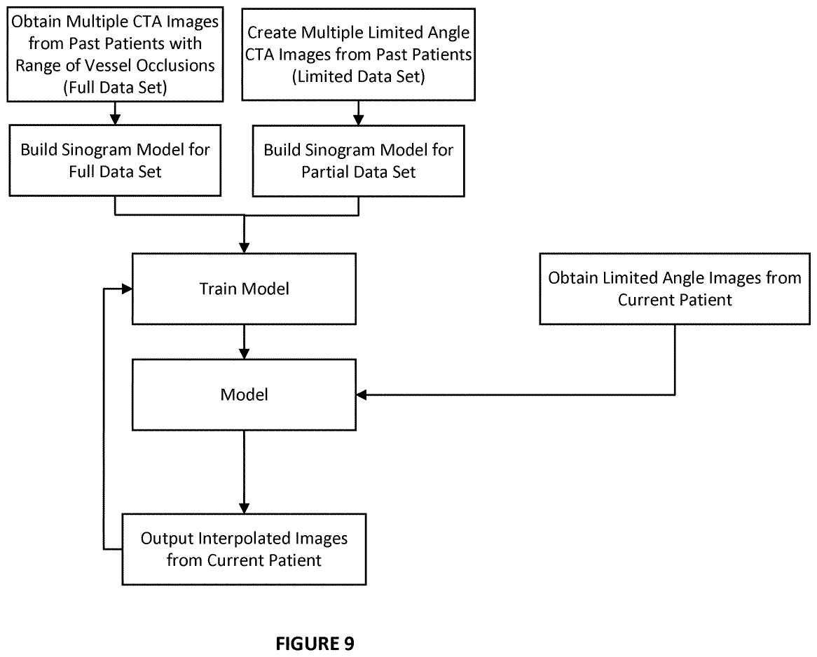

As shown in FIG. 9, using image data acquired from past patients (having a range of AIS conditions) together with a partial image data set from past patients can be utilized to build sinogram interpolation models.

For example, a past patient data set including raw data of a full range of projection angles can be filtered to create a limited data set for the same patient having limited projection angles. The data from the full data set and partial data set is used to then build sinogram models for each that are used to train an interpolation model.

After training, limited projection angle data is introduced into the model to provide interpolated output images for a current patient thus enabling diagnosis from the acquired limited projections

Although the present invention has been described and illustrated with respect to preferred embodiments and preferred uses thereof, it is not to be so limited since modifications and changes can be made therein which are within the full, intended scope of the invention as understood by those skilled in the art.

1. A method of obtaining a series of perfusion images of a brain of a patient suspected of suffering from acute ischemic stroke (AIS) and having been injected with contrast dye, the method comprising:
a) utilizing a neuroangiography machine having a first x-ray imaging system (FXIS) having a first x-ray emitter and a first x-ray receiver, the FXIS operatively positioned in a generally anterior/posterior position (AP) about the patient's head and a second x-ray imaging system (SXIS) having a second x-ray emitter and second x-ray receiver, the SXIS operatively positioned in a generally lateral position about the patient's head;
b) obtaining a series of images from the FXIS and SXIS wherein the FXIS is activated to alternately move between first and second imaging positions and the SXIS is activated to alternately move between third and fourth imaging positions;
c) repeating (b) over a plurality of time points between time=0 (t₀) to time=n (tₙ), wherein t₀ generally corresponds to contrast arrival in upper cervical vessels and tₙ generally corresponds to contrast washout; and
d) processing the images from (b) and (c) to create post-processed images showing a visualization of perfusion dynamics of the patient's brain.

2. The method of claim 1, wherein (d) includes processing combinations of images from the FXIS and SXIS as a pair of images obtained within a time threshold, and wherein if an area of affected tissue is detected, a relative depth of the affected tissue is estimated based on a stereoscopic offset analysis from a pair of images.

3. The method of claim 1, wherein (d) further includes introducing images from (c) into an interpolation model and activating the model to match the images to a past image database derived from patients having been diagnosed with acute ischemic stroke by past computed tomography images and utilizing best-fit images from the past image database to interpolate for a greater number of image angles to improve the resolution of the post-processed images.

4. The method of claim 1, wherein a first pair of images is acquired by activation of the FXIS to move to the first imaging position and subsequent movement of the FXIS to the second imaging position.

5. The method of claim 1, wherein a second pair of images is acquired by activation of the SXIS to move to the third imaging position and subsequent movement of the SXIS to the fourth imaging position.

6. The method of claim 1, wherein a first pair of images is acquired by activation of the FXIS to move to the first imaging position and movement of the SXIS to the second imaging position and images of the FXIS and SXIS are acquired simultaneously.

7. The method of claim 1, wherein a second pair of images is acquired by activation of the FXIS to move to the third imaging position and movement of the SXIS to the fourth imaging position and images of the FXIS and SXIS are acquired simultaneously.

8. The method of claim 1, wherein each of the first and second imaging positions and third and fourth imaging positions are about 15-30° apart.

9. The method of claim 1, wherein the post-processed images are analyzed to classify affected brain tissue based on relative perfusion of the affected brain tissue on a color scale showing a range of colors between fully perfused brain tissue and dead brain tissue.

10. The method of claim 1, wherein tₙ is 40-60 seconds.

11. A method of training and utilizing an image-interpolation model to improve resolution of a plurality of flat-panel x-ray images derived from limited projection angles in a current patient, the plurality of flat-panel x-ray images undergoing post-processing to assemble diagnostic images for diagnosis of acute ischemic stroke (AIS) in the current patient, the method comprising:
a) obtain a first plurality of computed tomography angiography (CT) images, the first plurality of CT images being defined as a full data set from a plurality of past patients having been diagnosed with AIS, wherein the first plurality of CT images include images from a plurality of projection angles;
b) obtain a second plurality of CT images, the second plurality of CT images being defined as a partial data set from a plurality of past patients having been diagnosed with AIS, wherein the second plurality of CT images include images from fewer projection angles than in (a);
c) build a sinogram model for the first plurality of CT images and the second plurality of CT images;
d) train an interpolation model using the first plurality of CT images and the second plurality of CT images to interpolate between projection angles of the second plurality of CT images based on determining a best-fit of a set of the second plurality of CT images to the first plurality of CT images; and
e) introduce a plurality of current patient flat-panel x-ray images into the model and analyze to determine a best-fit with the first plurality of CT images to create interpolated images from the current patient.

12. The method of claim 11, wherein the first plurality of CT images include images from more than 90 projection angles.

13. The method of claim 11, wherein the second plurality of CT images includes images from fewer than 10 projection angles.

14. A method of operating a neuroangiography machine having a first x-ray imaging system (FXIS) having a first x-ray emitter and a first x-ray receiver and a second x-ray imaging system (SXIS) having a second x-ray emitter and second x-ray receiver, comprising:
a) moving the FXIS to a first position in a generally anterior/posterior position relative to a patient's head;
b) moving the SXIS to a second position in a generally lateral position relative to a patient's head;
c) activating the FXIS to alternatively move between a first imaging position and a second imaging position through an arc about the anterior/posterior position;
d) simultaneously activating the SXIS to alternatively move between a third imaging position and fourth imaging position through an arc about the lateral position; and
e) obtaining x-ray images at each of the first, second, third and fourth imaging positions.

15. The method of claim 14, wherein movement of the FXIS and SXIS is coordinated to enable acquisition of images simultaneously from the FXIS and SXIS when the FXIS is at the first or second imaging position and the SXIS is at the third or fourth imaging position.

16. The method of claim 14, wherein (c) to (e) are repeated over a plurality of time points between time=0 (t₀) to time=n (tₙ), and wherein after injection of contrast agent into the patient, tₙ generally corresponds to contrast arrival in upper cervical vessels and tₙ generally corresponds to contrast washout.

17. The method of claim 14, wherein each of the first and second imaging positions and third and fourth imaging positions are about 15-30° apart.

18. The method of claim 14 further comprising post-processing images to classify affected brain tissue based on relative perfusion of the affected brain tissue on a scale discerning between fully perfused brain tissue and dead brain tissue.

19. The method of claim 14, wherein tₙ is 40-60 seconds.
